(19) United States
(12) Patent Application Publication
(10) Pub. No.: US 2003/0053975 A1
(43) Pub. Date: Mar. 20, 2003

(54) W/O EMULSIONS COMPRISING AMINO-FUNCTIONAL ORGANOPOLYSILOXANES

(76) Inventors: INGRID EISSMANN, GELSENKIRCHEN (DE); JUTTA HENNING, ESSEN (DE); FELIX MULLER, VELBERT (DE); STEFAN STADTMULLER, ESSEN (DE)

Correspondence Address: FROMMER LAWRENCE & HAUG, 745 FIFTH AVENUE- 10TH FL., NEW YORK, NY, 10151, US

(21) Appl. No.: 09/457,449

(22) Filed: Dec. 8, 1999

(30) Foreign Application Priority Data
Dec. 10, 1998 (DE) 198 56 930.0

(51) Int. Cl. A61K 7/06; A61K 7/11

(52) U.S. Cl. 424/70.12

(57) ABSTRACT

The invention relates to W/O emulsions comprising amino-functional organopolysiloxanes and constituting car polishes, cleaning products for vitro ceramic hobs, textile treatment compositions, shoecare compositions, cosmetics, and hair gel.

### RELATED APPLICATIONS

This application claim priority to German application No. 198 56 930.0, filed on Dec. 12, 1998, herein incorporated by reference.

### BACKGROUND OF THE INVENTION

The invention relates to W/O emulsions comprising amino-functional organopolysiloxanes.

### FIELD OF THE INVENTION

The invention relates to W/O emulsions comprising amino-functional organopolysiloxanes.

### DESCRIPTION OF THE RELATED ART

DE 44 24 914 A1 relates to amino-functional organopolysiloxanes containing groups of the general formula (III)

—R²—NR³/CH₂)ₘNR⁴R⁵

where

R² is a divalent C₁ to C₁₈ hydrocarbon radical,

R³ is a hydrogen atom or an unsubstituted or fluoro-, chloro- or bromo-substituted C₁ to C₁₀ alkyl radical,

R⁵ is an unsubstituted or fluoro-, chloro- or bromo-substituted C₁ to C₁₀ alkyl radical, and

m is 2,3 or 4.

The amino-functional organopolysiloxanes can be used to treat organic fibers.

EP 0 450 656 A1 relates generally to car polishes and their components.

The prior art primarily discloses O/W emulsions, which are used to produce car polish, cleaning products for vitro ceramic hobs, textile treatment compositions, shoecare compositions, cosmetics, or hair gel. A disadvantage of the O/W emulsion is that it can be rinsed off by re-emulsion owing to the passage of water or moisture onto the treated surface.

### BRIEF DESCRIPTION OF THE FIGURE

FIG. 1 depicts a graph that compares the weathering of a metal car panel which has been treated with a polish of the present invention against a commercially available car polish.

### OBJECT OF THE INVENTION

In contrast, an object of the present invention is to provide stable emulsions which in contradistinction to the prior art are insensitive to re-emulsion as a result of renewed contact with water or moisture.

### SUMMARY OF THE INVENTION

This object is achieved by means of a W/O emulsion comprising amino-functional organopolysiloxanes of the general formula (I)

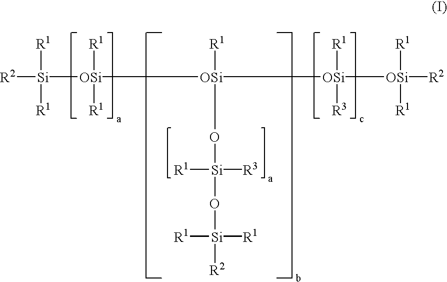

where

a is from 1 to 500 and

b is from 0 to 50,

c is from 1 to 50,

and the radicals

R¹ are identical or different aliphatic or aromatic hydrocarbon radicals, preferably having 1 to 8 carbon atoms, and

R² are R¹ or a hydroxyl or alkoxy group —O—R¹, with the proviso that at least one radical R² is the radical —OH or —O—R¹,

R³ are a group of the general formula

—[(R⁴)_{d}—NR⁵]ₑ—R⁵,

where

R⁴ is a divalent hydrocarbon radical, preferably a C₁ to C₁₂ hydrocarbon radical, or is an —OCH₂CH₂— or an —OCH₂CH(CH₃)— unit,

R⁵ is a divalent hydrocarbon radical, preferably a C₁ to C₁₂ hydrocarbon radical, or a hydrogen atom, and

d is 1 to 10, and

e is 1 to 5.

### DESCRIPTION OF PREFERRED EMBODIMENTS

When using the amino-functional organosiloxanes of the general formula (I) there is generally no need for further emulsifiers. In many cases these compounds simultaneously constitute active substance and emulsifier, and so the concentration of other components can be reduced. The amino-functional organopolysiloxanes used in accordance with the invention therefore function as self-emulsifiers. An important feature of the emulsions is the W/O format, which in relation to the O/W emulsions known in the prior art, has the major advantage of longer weathering stability. Compared with products on a O/W basis that are available commercially, a car polish or wax preservative therefore remains longer on the paint surface when the latter is exposed to a humid environment.

Depending on preparation conditions and raw materials used, a large number of amino-functional organopolysiloxanes of the general formula (I) can be prepared. The general process for their preparation is known from DE 44 24 914 A1, the full content of which is incorporated herein by reference.

For the purposes of the present invention it is particularly preferable for the indices in the general formula (I) to have the following values:

a is from 50to 250,

b is from 0 to 10 and

c is from 1 to 20.

Particular advantages result for emulsions defined in this way since even low concentrations of the amino-functional organopolysiloxane lead to high emulsion stability.

Furthermore, particular preference is given in accordance with the invention to emulsions in which in the general formula (I)

R¹ is a methyl radical,

R⁴ is a C₁ to C₆ hydrocarbon radical,

R⁵ is a C₁ to C₆ hydrocarbon radical,

d is from 1 to 3, and

e is from 1 to 3.

Particular advantages in terms of stability apply to the emulsions obtainable using the amino-functional organosiloxanes defined in this way.

In the W/O emulsions of the invention, the amount of the amino-functional organopolysiloxanes of the general formula (I) can be varied within a wide range. For the purposes of the present invention it is particularly preferred for said emulsions to contain from about 1 to about 60% by weight of said organopolysiloxanes, depending on the field of application. If the amount of organopolysiloxanes of the general formula (I) is established at too low a level, a sufficient emulsifying effect will not be in evidence. On the other hand, excessive concentrations of emulsifier should be avoided, since it would not be possible to incorporate any further raw materials and the limit of what is economically acceptable would be breached.

With particular preference in the context of the present invention, the emulsions contain no emulsifiers other than the amino-functional organopolysiloxanes of the general formula (I). In this case, in particular, the organopolysiloxanes act simultaneously as active substance and emulsifier.

The emulsions of the invention embrace, in particular, car polishes, cleaning products for vitro ceramic hobs, textile treatment compositions, shoecare compositions, cosmetics, and hair gels. Besides the amino-functional organopolysiloxanes of the general formula (I), the emulsions use further components which are known per se in the prior art, examples include cationic surfactants, oils, abrasives, waxes, conditioning agents, colorants, and odorants.

A particularly preferred car polish contains, for example, from 1 to 10% by weight, in particular from 3 to 5% by weight, of the amino-functional organopolysiloxanes of the general formula (I). Similarly, a cleaning product for vitro ceramic should contain from 3 to 20% by weight, in particular from 8 to 15% by weight, of the amino-functional organopolysiloxanes of the general formula (I) along with further components which are known per se, especially abrasives and waxes.

### WORKING EXAMPLES

### a) Preparation Examples

### Example 1

44 g (0.23 mol) of γ-aminopropyl-N-aminoethyldimethoxymethylsilane, methylsilicone fluid (Tegiloxan® 1000) (1000 g) and KOH (as a 20% solution in ethanol; 50 ml) were heated to 90° C. in a 2 1 reaction vessel and allowed to react at this temperature for 5 hours. The catalyst was then deactivated by adding equimolar amounts of sodium bicarbonate and the mixture was held at the temperature for a further 4 hours. Subsequently, volatile components were separated off by applying reduced pressure (20-30 mbar) and briefly raising the temperature to 120° C.

Filtration gave a colorless liquid having a viscosity of about 100 mPas [25° C.] which according to ¹H and ²⁹Si NMR spectra is assigned the following formula

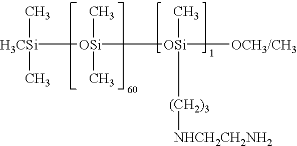

The amine content was 0.6% by weight.

### Example 2

40 g (0.21 mol) of γ-aminopropyldiethoxymethylsilane, methylsilicone fluid (Tegiloxan® 1000) (1000 g) and tetramethylammonium hydroxide pentahydrate (0.05 g) were heated to 90° C. in a 2 l reaction vessel and allowed to react at this temperature for 10 hours. The catalyst was then deactivated under reduced pressure (10-20 mbar) at 150° C. for 2 hours, with simultaneous distillative removal of volatile components, after which the mixture was filtered.

NMR data for the colorless liquid having a viscosity of about 80 mPas [25° C] were in accord with the following structure

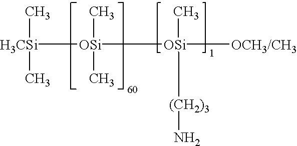

The amine content was 0.3% by weight.

For the subsequent examples, especially the glass ceramic and autocare compositions, there were no additives, such as emulsifiers and thickeners.

### USE EXAMPLES

### Example 3

Care emulsion for vitro ceramic hobs

15.0% aminosiloxane from Example 1 (0.6% N)

5.0% decamethylcyclopentasiloxane (TEGO® Polish Additive 5)

10.0% methylsilicone fluid (TEGILOXAN®350)

20.0% abrasive (Sillitin®N 85 or Alcoa®P 15)

50.0% water

In this formulation it was possible to omit the thickener, which usually gave a fairly poor result in the “fingerprint”.

### Comparative Example 1

Care emulsion for vitro ceramic hobs (standard)

38.0% nonionic amino-functional silicone fluid emulsion containing 40% of an aminosiloxane (1% N), 2% of PEG-20 glyceryl laurate, 6% of cocamidopropyl betaine, and 52% of water

1.0% ester wax

38.8% water

15.0% abrasive (Sillitin®N 85 or Alcoa®P 15)

0.2% thickener (Carbomer® 140)

2.0% lemon oil

Assessment of the protective effect by means of the “sugar test” in direct comparison:

Test Method:

This laboratory test was developed in order to enable the individual active substances to be differentiated and thus assessed in terms of their protective effect.

Procedure:

1) A vitro ceramic laboratory protection plate (SCHOTT), 155×155 mm, was cleaned with alcohol;

2) the plate was divided into the corresponding test fields (max. 4) using adhesive tape;

3) the active substances or formulations to be tested were applied and polished out;

4) the adhesive tape was removed;

5) assessment of smoothness, gloss and ease of rubout;

6) a hotplate was heated on full power and, for better heat transfer, a metal plate likewise measuring 155×155 mm was placed on top;

7) the vitro ceramic plate to be tested was placed on the heating apparatus and allowed to reach maximum temperature;

8) then, about ½ a teaspoonful of sugar was placed in each test field;

9) after the sugar had melted and began to caramelize, the hotplate was switched off;

10) after the vitro ceramic plate had cooled, the sugar was carefully lifted off. Even at this stage, initial differences were evident.

Sugar Test:

Assessment of the formulations in direct comparison:

### Example 3

| | |
|---|---|
| Comparative Example 1: | |
| Comparative Example 2: | (commercial cleaner for ceramic hob plates) |
| Comparative Example 3: | (commercial care composition for ceramic hob |
| | plates) |

1) Sugar Removal:

| | |
|---|---|
| easy: | rating 1 |
| difficult: | rating 3 |
| impossible (without auxiliary means) | rating 5 |

Result: Average of 5 Comparative Measurements:

| | |
|---|---|
| Example 3: | 1.0 |
| Comparative Example 1: | 1.4 |
| Comparative Example 2: | 3.4 |
| Comparative Example 3: | 1.8 |

2) Residue on the Vitro Ceramic Field:

| | |
|---|---|
| very good: | virtually no residue or easily removable (1) |
| good: | removable with a wet sponge and detergent (2) |
| satisfactory: | removable with scouring milk (3) |
| adequate: | removable with appropriate scraper (4) |
| deficient: | removable only with the aid of combined cleaning |
| | measures (5) |
| unsatisfactory: | impossible to remove (6) |

Result: Average of 5 Comparative Measurements:

| | |
|---|---|
| Example 3: | 1.4 |
| Comparative Example 1: | 2.4 |
| Comparative Example 2: | 4.3 |
| Comparative Example 3: | 2.8 |

5 measurements were carried out for each formulation. For each measurement a new glass ceramic field was treated only once with the corresponding formulation in order to avoid falsification as a result of a cumulative effect (residue of care composition).

In a quality comparison of the formulation of the invention with the commercially available products, measured in terms of gloss, care and protection against the burning-in of sugary foods, it was found that all three properties are very good. Particularly noteworthy is the protection against burning-in of sugar, since in this respect the formulation of the invention is markedly superior to the commercial products.

General advantages of the formulation of the invention can be seen in the fact that the use of thickeners was unnecessary. The abrasive materials showed no settling.

Since the formulation of the invention was a W/O system, it was not re-emulsifiable by water (wet wiping) following application.

### Example 4

Car polish, detergent-resistant

| | |
|---|---|
| 10.0% | aminosiloxane (from Example 1) |
| 15.0% | aromatic-free mineral spirit (Shellsol ® TD), |
| | boiling range 172 to 185° C. (ASTM D 1078) |
| 63.0% | water |
| 5.0% | methylsilicone fluid (TEGILOXAN ® 350) |
| 2.0% | Camauba wax |
| 5.0% | abrasive (Sillitin ® N 85 or Alcoa ® P 15) |

### Comparative Example 4

Car polish, detergent-resistant O/W

| | |
|---|---|
| 33.3% | commercial ester wax emulsion, 12% (Hoechst) |
| 20.0% | commercial aminosiloxane emulsion, containing |
| | 35% of aminosiloxane, 10% of cocamidopropyl |
| | betaine, 2% of PEG-20 glyceryl laurate, and |
| | 53% of water (TEGO ® Polish Additive |
| | (11/59 emulsion: aminosiloxane 7% |
| | active-ingredient content)) |
| 41.5% | water |
| 5.0% | abrasive (Sillitin ® N 85 or Alcoa ® P 15) |
| 0.2% | commercial thickener (Carbomer ® 140) |

The comparison of Example 4 with Comparative Example 4 was carried out and assessed in accordance with “Test 2/86” and “quality standards for car care and cleaning products”, “SÖFW/June 78”. The results of this comparison are summarized in FIG. 1.

A used painted metal car panel was cleaned and treated with the auto polishes to be tested. The prepared test panel was weathered for 6 weeks. During this time, the test panel was washed with shampoo at regular intervals (2×per week). The following criteria were assessed: detergent resistance, gloss, water repellence, and water beading effect.

After weathering and the twelve washes, the area treated in accordance with the invention showed the best long-term protection.

In comparison with commercially available products, the resistance to detergent from 12 washes with shampoo, the stability of gloss, the water repellency, and the water beading effect were assessed over a period of 6 weeks' outdoor weathering in the above long-term test procedure.

Car polishes containing only silicone fluid showed a marked loss in quality after about 5 wash cycles.

Car polishes which came through the prescribed number of 12 wash cycles with a really good result contained 5% aminosiloxane.

The result for the car polish of the invention was excellent. Here again, it was found that the W/O system was not re-emulsifiable.

The above description of the invention is intending to be illustrative and not limiting. Various changes or modification in the embodiments described may occur to those skilled in the art. These changes can be made without departing from the scope or spirit of the invention.

What is claimed is:

1. A W/O emulsion comprising amino-functional organopolysiloxanes of the formula (I) 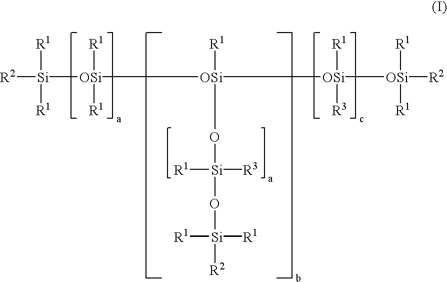
where
a is from 1 to 500 and
b is from 0to 50,
c is from 1 to 50,
and the radicals
R¹ are identical or different aliphatic or aromatic hydrocarbon radicals, and
R² are R¹ or a hydroxyl or alkoxy group —O—R¹, with the proviso that at least one radical R² is the radical —OH or —O—R¹,
R³ are a group of the general formula
—[(R⁴)_{d}—NR⁵]ₑ—R⁵,
where
R⁴ is a divalent hydrocarbon radical or is an —OCH₂CH₂— or an —OCH₂CH(CH₃)— unit,
R⁵ is a divalent hydrocarbon radical or a hydrogen atom, and
d is 1 to 10, and
e is 1 to 5.

2. An emulsion as claimed in claim 1 where, in the radicals the amino functional organiopolysiloxane, the radicals
R¹ are identical or different aliphatic or aromatic hydrocarbon radicals having 1 to 8 carbon atoms, and
R¹ are R¹ or a hydroxyl or alkoxy group —O—R¹, with the provision that at least one radical R² is the radical —OH or —OR¹,
R³ are a group of the general formula —[(CR⁴)_{d}—NR⁵]ₑ—R⁵,
where
R⁴ is a divalent C₁ to C₁₂ hydrocarbon radical or is —OCH₂CH₂— or and —OCH₂CH(CH₃)— units,
R⁵ is a divalent C₁ to C₁₂ hydrocarbon radical or a hydrogen atom.

3. An emulsion as claimed in claim 1, wherein the indices of the formula (I) have the following meanings:
a is from 50 to 250,
b is from 0 to 10, and
c is from 1 to 20.

4. An emulsion as claimed in claim 1, wherein, in the formula (I)
R¹ is a methyl radical,
R⁴ is a C₁ to C₆ hydrocarbon radical,
R⁵ is a C₁ to C₆ hydrocarbon radical,
d is from 1 to 3, and
e is from 1 to 3.

5. An emulsion as claimed in claim 2, where, in the amino-functional organopolysiloxone,
a is from 50 to 250;
b is from 20 to 10;
c is from 1 to 20;
R¹ is a methyl radical
R² is a C₁ to C₂ hydrocarbon radical;
R⁵ is a C₁ to C₂ hydrocarbon radical;
d is from 1 to 3, and
e is from 1 to 3.

6. An emulsion as claimed claim 1, which comprises from about 1 to about 60% by weight of amino-functional organosiloxanes of the formula (1).

7. An emulsion as claimed in claim 1, which comprises no further emulsifiers.

8. An emulsion as claimed in claim 1 which comprises from 1 to 60% by weight of an amino-organosiloxane of claim 2.

9. The emulsion as claimed in claim 8, which does not further comprise an additional emulsifer.

10. An emulsion according to claim 1, where the amino-functional organipolysiloxane is

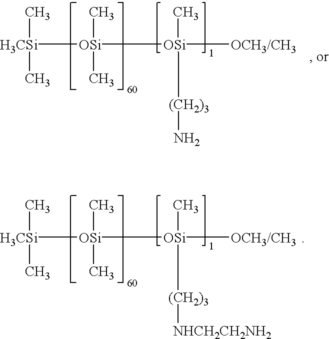

11. An emulsion according to claim 1 which further comprises a cosmetic surfactant, oil, abrasive, wax, conditioning agent, colorant or deodorant.

12. A car polish which comprises an emulsion according to claim 1.

13. A cosmetic which comprises an emulsion according to claim 1.

14. A hair gel which comprises an emulsion according to claim 1.

15. A shoecare composition which comprises an emulsion according to claim 1.

16. A textile treatment composition which comprises an emulsion according to claim 1.

17. A composition for cleaning vitro ceramic hobs which further comprises an emulsion according to claim 1.
